(19) United States
(12) Patent Application Publication
(10) Pub. No.: US 2019/0110945 A1
(43) Pub. Date: Apr. 18, 2019

(54) Massage Machine

(71) Applicant: Fuji Medical Instruments Mfg. Co., Ltd., Osaka (JP)

(72) Inventors: Nobukazu KAWAGOE, Osaka (JP); Masafumi TAKARABE, Osaka (JP)

(73) Assignee: Fuji Medical Instruments Mfg. Co., Ltd., Osaka (JP)

(21) Appl. No.: 16/044,300

(22) Filed: Jul. 24, 2018

(30) Foreign Application Priority Data
Oct. 17, 2017 (JP) 2017-201210

(51) Int. Cl. A61H 7/00; A61H 15/00; A61B 5/00

(52) U.S. Cl. CPC A61H 7/007; A61H 15/0078; A61H 2201/0149; A61H 2201/1207; A61B 5/0053; A61B 5/4519

(57) ABSTRACT

A massage mechanism includes a portion, which, in a muscle hardness measurement mode, drives an advancing/retreating motor to rotate in a predetermined direction by constant-speed control to press a massage element into a body of a person to be treated, a portion monitoring a change rate of a motor current flowing through the advancing/retreating motor during driving of the advancing/retreating motor and stopping the driving of the advancing/retreating motor when the change rate of the motor current becomes not less than a predetermined threshold, a portion, which, after stoppage of the driving of the advancing/retreating motor, drives a tapping motor to rotate by constant-speed control to make the massage element perform a tapping operation, a current detection portion, detecting a motor current flowing through the tapping motor during driving of the tapping motor, and a portion measuring a muscle hardness of the person to be treated based on the motor current detected by the current detection portion.

### RELATED APPLICATIONS

This application claims priority to Japanese Patent Application No. 2017-201210, filed Oct. 17, 2017, which is hereby incorporated herein by reference in its entirety.

### BACKGROUND OF THE INVENTION

### 1. Field of the Invention

The present invention relates to a massage machine.

### 2. Description of the Related Art

Japanese Patent Application Publication No. 2010-104525 discloses a muscle hardness meter arranged to measure a hardness of a muscle (muscle hardness) accurately by reducing influences of differences in subcutaneous fat thickness among human bodies.

### SUMMARY OF THE INVENTION

The inventor of preferred embodiments of the present invention described and claimed in the present application conducted an extensive study and research regarding a massage machine, such as the one described above, and in doing so, discovered and first recognized new unique challenges and previously unrecognized possibilities for improvements as described in greater detail below.

It would be convenient if a massage machine can be made to have a function of measuring a muscle hardness of a person to be treated because the function can be used to check the massage effect, etc. It may be thus be considered to mount a muscle hardness meter such as described in Japanese Patent Application Publication No. 2010-104525 to a backrest portion of a massage machine. However, with such a massage machine, there is a problem in that a special muscle hardness meter is required separately, causing the arrangement to become complex and the cost to be high.

An object of the present invention is to provide a massage machine that enables a hardness of just a muscle of a person to be treated to be measured without using special measurement equipment.

In order to overcome the previously unrecognized and unsolved challenges described above, a preferred embodiment of the present invention provides a massage machine. The massage machine is a massage machine having a muscle hardness measurement mode of measuring a muscle hardness of a person to be treated and includes a massage element driving unit, having a first electric motor as a driving source and including a massage mechanism arranged to make a massage element perform a massage operation, a moving mechanism, having a second electric motor as a driving source and arranged to move the massage element driving unit in an approaching/separating direction with respect to a body of the person to be treated, a second electric motor driving portion, which, in the muscle hardness measurement mode, drives the second electric motor to rotate in a predetermined direction by constant-speed control to press the massage element into the body of the person to be treated, a second electric motor drive stopping portion, monitoring a change rate of a motor current flowing through the second electric motor during driving of the second electric motor by the second electric motor driving portion and stopping the driving of the second electric motor when the change rate of the motor current becomes not less than a predetermined threshold, a first electric motor driving portion, which, after stoppage of the driving of the second electric motor by the second electric motor drive stopping portion, drives the first electric motor to rotate by constant-speed control to make the massage element perform the massage operation, a current detection portion, detecting a motor current flowing through the first electric motor during driving of the first electric motor by the first electric motor driving portion, and a muscle hardness measurement portion, measuring the muscle hardness of the person to be treated based on the motor current detected by the current detection portion.

With the present arrangement, in the muscle hardness measurement mode, after the massage element is pressed in by the second electric motor to a position at which the skin and fat of the person to be treated cannot be compressed any further, the first electric motor can be driven to rotate by constant-speed control to perform the massage operation using the massage element. The muscle hardness of the person to be treated can then be measured based on the motor current of the first electric motor detected during the massage operation. It is thereby made possible to measure the hardness of just the muscle.

The first electric motor is the driving source of the massage mechanism, arranged to make the massage element perform the massage operation, and the second electric motor is the driving source of the moving mechanism, arranged to move the massage element driving unit, including the massage mechanism, in the approaching/separating direction with respect to the person to be treated. That is, the first electric motor and the second electric motor are motors that are inherently included in the massage machine for performing massage on the person to be treated. Therefore, with the present arrangement, the hardness of just the muscle can be measured without using special measurement equipment.

In the preferred embodiment of the present invention, the muscle hardness measurement portion is arranged to calculate, as an index value of the hardness of the muscle of the person to be treated, an average value of the motor current detected by the current detection portion in a predetermined time during the driving of the first electric motor by the first electric motor driving portion.

In the preferred embodiment of the present invention, the massage mechanism is a tapping mechanism arranged to make the massage element perform a tapping operation.

In the preferred embodiment of the present invention, the massage mechanism is a kneading mechanism arranged to make the massage element perform a kneading operation.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a partially cutaway perspective view of an outer appearance of a chair type massage machine according to a preferred embodiment of the present invention.

FIG. 2 is an illustrative perspective view of an arrangement of a massage unit.

FIG. 3 is a block diagram of an electrical configuration of the chair type massage machine.

FIG. 4 is a block diagram of arrangements of a driving circuit of an advancing/retreating motor and an advancing/retreating motor controller and showing an advancing/retreating motor current calculating portion.

FIG. 5 is a block diagram of arrangements of a driving circuit of a raising/lowering motor and a raising/lowering motor controller.

FIG. 6 is a block diagram of arrangements of a driving circuit of a kneading motor and a kneading motor controller and showing a kneading motor current calculating portion.

FIG. 7 is a block diagram of arrangements of a driving circuit of a tapping motor and a tapping motor controller and showing a tapping motor current calculating portion.

FIG. 8 is a flowchart of procedures of a muscle hardness measurement process executed by a muscle hardness measurement process portion.

FIG. 9 is a graph of change with time of a motor current flowing through the advancing/retreating motor when massage elements are pressed into a body at a constant speed by the advancing/retreating motor.

FIG. 10 is a flowchart of procedures of another example of the muscle hardness measurement process executed by the muscle hardness measurement process portion.

### DETAILED DESCRIPTION OF THE PREFERRED EMBODIMENTS

A preferred embodiment of a case of applying the present invention to a chair type massage machine shall now be described in detail with reference to the attached drawings.

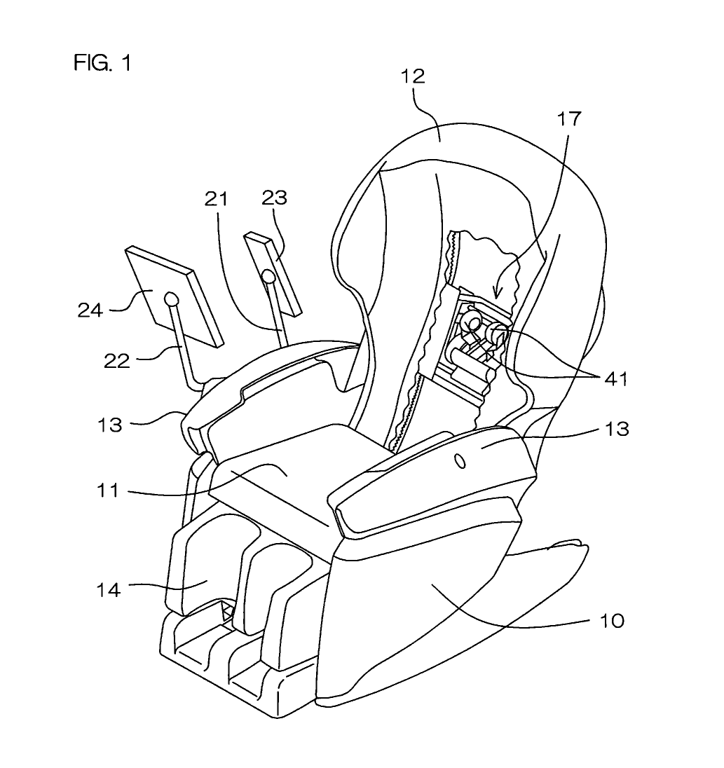

FIG. 1 is a partially cutaway perspective view of an outer appearance of the chair type massage machine 1 according to the preferred embodiment of the present invention.

The chair type massage machine 1 includes a seat portion 11, a backrest portion 12, armrest portions 13, a footrest portion (ottoman) 14, and a base portion 10, serving as a foundation for these portions.

In the description that follows, a front/rear direction, a right/left direction, and an up/down direction shall respectively refer to a front/rear direction, a right/left direction, and an up/down direction as viewed by a person to be treated when the person to be treated is seated in an ordinary orientation on the chair type massage machine 1.

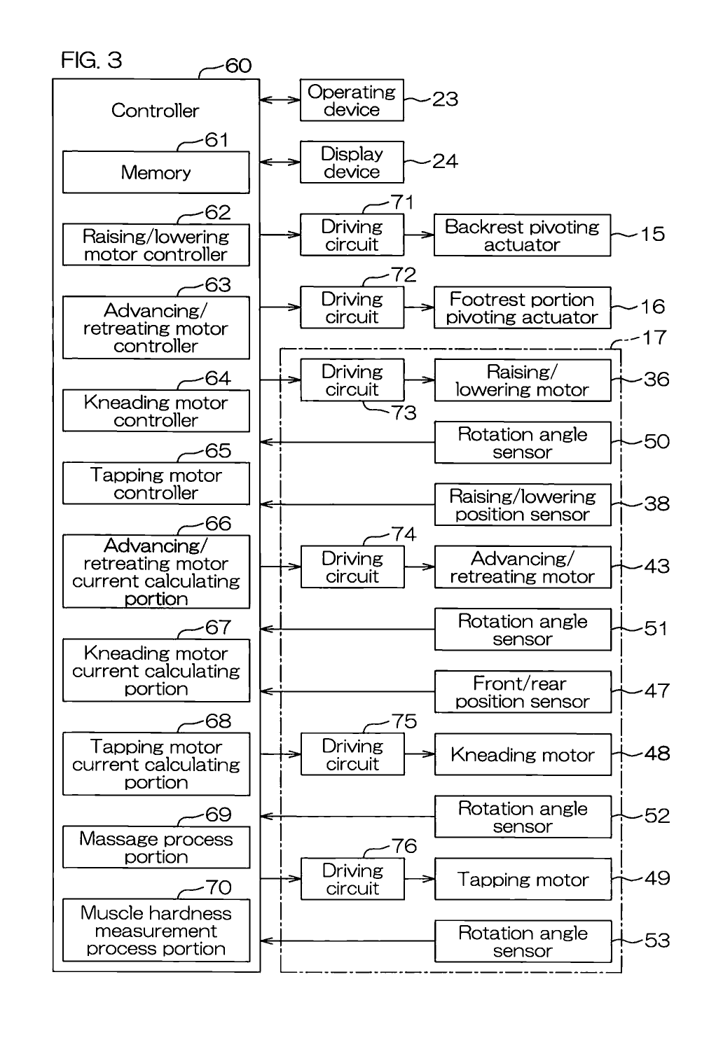

The seat portion 11 is disposed on the base portion 10. The backrest portion 12 is disposed at a rear of the seat portion 11. The armrest portions 13 are disposed at both right and left sides of the seat portion 11. The footrest portion 14 is disposed at a front side of the seat portion 11. The backrest portion 12 is supported by a backrest pivoting actuator 15 (see FIG. 3) such as to be tiltable with respect to the seat portion 11. Also, the footrest portion 14 is enabled to be pivoted around a support shaft, provided at a vicinity of a seat portion upper portion and extending in the right/left direction, as a center by a footrest portion pivoting actuator 16 (see FIG. 3).

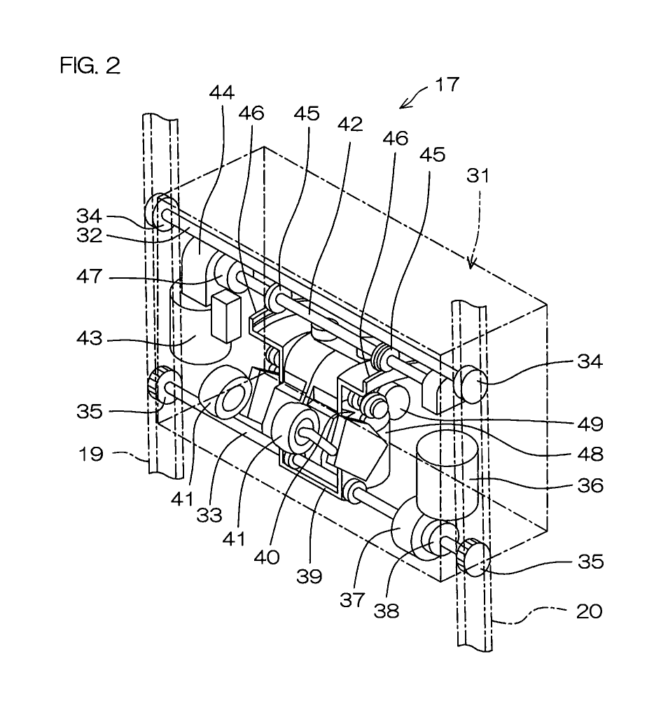

A massage unit 17 is incorporated in the backrest portion 12. The massage unit 17 is arranged to perform various types of massage using a pair of right and left massage elements (kneading balls) 41. The backrest portion 12 is provided with a pair of right and left guide rails 19 and 20 (see FIG. 2) of U-shaped cross section extending in the up/down direction, and the massage unit 17 is enabled to move in the up/down direction along the guide rails 19 and 20. A detailed arrangement of the massage unit 17 shall be described later.

The seat portion 11, the backrest portion 12, the armrest portions 13, and the footrest portion 14 are provided with airbags (not shown). Each airbag is inflated by being supplied with air from an air pump (not shown) via a solenoid valve (not shown). Each air bag has a flat form when deflated and is suitably inflated to apply a pressing stimulus to the person to be treated.

An operating device supporting member 21 and a display device supporting member 22 are mounted to one of the armrest portions 13. An operating device 23, for operation of the chair type massage machine 1 by the person to be treated, is detachably mounted to the operating device supporting member 21. A display device 24 is detachably mounted to the display device supporting member 22. The display device 24 is, for example, a liquid crystal display.

FIG. 2 is an illustrative perspective view of the arrangement of the massage unit 17.

The massage unit 17 is mounted vertically movably mounted to the guide rails 19 and 20. The massage unit 17 includes a main frame 31 of rectangular frame shape. The main frame 31 is constituted of a pair of right and left side walls and a top wall and a bottom wall respectively joining mutual upper ends and mutual lower ends of the side walls. A guide shaft 32 and a raising/lowering driveshaft 33 that extend in the right/left direction are rotatably mounted to the main frame 31. The guide shaft 32 is disposed at an upper portion of the main frame 31 and the raising/lowering driveshaft 33 is mounted to a lower portion of the main frame 31. Both end portions of the guide shaft 32 and the raising/lowering driveshaft 33 project outward from both side walls of the main frame 31. Guide rollers 34, guided by the guide rails 19 and 20, are mounted to both end portions of the guide shaft 32. Pinion gears 35, engaging with racks (not shown) provided in the guide rails 19 and 20, are mounted to both end portions of the raising/lowering driveshaft 33. A raising/lowering motor 36, arranged to rotate the raising/lowering driveshaft 33, is mounted to the main frame 31. The raising/lowering motor 36 is coupled to the raising/lowering driveshaft 33 via a gear mechanism 37. The massage unit 17 is raised and lowered along the guide rails 19 and 20 by the raising/lowering motor 36 being rotated.

The massage unit 17 is provided with a raising/lowering position sensor 38 arranged to detect a rotation amount of the raising/lowering driveshaft 33 to detect a raising/lowering position (up/down direction position) of the massage unit 17. The raising/lowering position sensor 38 is constituted of a rotary encoder arranged to detect the rotation amount of the raising/lowering driveshaft 33.

At an intermediate portion of a length of the raising/lowering driveshaft 33, a swinging frame 39 is mounted such as to be swingable in the front/rear direction. A pair of right and left massage elements 41 and a massage element driving unit 40, including a driving mechanism therefor, are mounted to the swinging frame 39. The main frame 31 is provided with a massage element driving unit advancing/retreating mechanism (moving mechanism) arranged to make the swinging frame (massage element driving unit) advance and retreat in the front/rear direction.

The massage element driving unit advancing/retreating mechanism shall now be described. An advancing/retreating shaft 42, disposed at a lower side of the guide shaft 32 and extending in the right/left direction, is rotatably mounted to the main frame 31. An advancing/retreating motor 43, arranged to rotate the advancing/retreating shaft 42, is mounted to a side portion at one side of the main frame 31. The advancing/retreating motor 43 is coupled to the advancing/retreating shaft 42 via a gear mechanism 44. A pair of right and left pinion gears 45 are mounted to intermediate portions of a length of the advancing/retreating shaft 42. Arcuate racks 46, engaging with the pair of right and left pinion gears 45, are respectively provided at upper portions of the swinging frame 39. When the advancing/retreating shaft 42 is rotated by the advancing/retreating motor 43, the pinion gears 45 rotate and the arcuate racks 46 move. Thereby, the swinging frame 39 swings around the raising/lowering driveshaft 33 as a center. Thereby, the massage element driving unit 40 (massage elements 41) advances and retreats in the front/rear direction.

The massage unit 17 is provided with a front/rear position sensor 47 arranged to detect a rotation amount of the advancing/retreating shaft 42 to detect a front/rear position of the massage element driving unit 40. The front/rear position sensor 47 is constituted of an encoder arranged to detect the rotation amount of the advancing/retreating shaft 42.

The massage element driving unit 40 includes a kneading mechanism that eccentrically pivots the massage elements 41 to perform a kneading operation and a tapping (beating) mechanism that swings the massage elements 41 back and forth to perform a tapping (beating) operation. The kneading mechanism or the tapping mechanism is an example of a massage mechanism. The kneading mechanism includes a kneading motor 48 as an actuator (driving source). The tapping mechanism includes a tapping motor 49 as an actuator (driving source). With the kneading operation, there is “kneading up,” in which the massage elements operate in the up direction, and “kneading down,” in which the massage elements operate in the down direction. Between “kneading up” and “kneading down,” a rotation direction of the kneading motor 48 is reversed.

The massage unit 17 is provided with a rotation angle sensor 50 (see FIG. 3), arranged to detect a rotation angle of a rotor of the raising/lowering motor 36, and a rotation angle sensor 51 (see FIG. 3), arranged to detect a rotation angle of a rotor of the advancing/retreating motor 43. Also, the massage unit 17 is provided with a rotation angle sensor 52 (see FIG. 3), arranged to detect a rotation angle of a rotor of the kneading motor 48, and a rotation angle sensor 53 (see FIG. 3), arranged to detect a rotation angle of a rotor of the tapping motor 49. Each of the rotation angle sensors 50 to 53 is constituted, for example, of a resolver or encoder, etc.

FIG. 3 is a block diagram of an electrical configuration of the chair type massage machine 1. In FIG. 3, the air pump, a driving circuit therefor, the solenoid valve, and a driving circuit therefor that are arranged to inflate and deflate each air bag are omitted for convenience of description.

A controller 60, arranged to control the chair type massage machine 1, is incorporated in an interior of the chair type massage machine 1. The controller 60 includes a microcomputer and includes a CPU and a memory (RAM, ROM, or nonvolatile memory) 61, etc. A program for controlling the chair type massage machine 1 and necessary data, etc., are stored in the memory 61. The controller 60 includes an internal clock.

The operating device 23, the display device, 24, a driving circuit 71 of the backrest pivoting actuator 15, and a driving circuit 72 of the footrest portion pivoting actuator 16 are connected to the controller 60. Further, a driving circuit 73 of the raising/lowering motor 36, a driving circuit 74 of the advancing/retreating motor 43, a driving circuit 75 of the kneading motor 48, and a driving circuit 76 of the tapping motor 49 inside the massage unit 17 are connected to the controller 60. Further, the raising/lowering position sensor 38, the front/rear position sensor 47, and the rotation angle sensors 50, 51, 52, and 53 inside the massage unit 17 are connected to the controller 60.

The controller 60 controls the driving circuits 71 and 72 of the respective actuators 15 and 16 based on operations of the operating device 23, etc. Also, the controller 60 controls the driving circuits 73 to 76 of the respective motors 36, 43, 48, and 49, the driving circuit (not shown) of the air pump, and the driving circuit (not shown) of the solenoid valve based on operations of the operating device 23, etc. The chair type massage machine 1 is thereby made capable of performing the various types of massage and measurement of a muscle hardness (a hardness of a muscle).

In other words, the chair type massage machine 1 has a massage mode and a muscle hardness measurement mode. With the massage mode, there is an automatic mode and a manual mode. In the automatic mode, massage is performed in accordance with a massage course selected from among a plurality of types of massage courses by the person to be treated. In the manual mode, the massage is performed in accordance with a massage type selected by the person to be treated. In the muscle hardness measurement mode, a muscle hardness measurement process for measuring the muscle hardness of the person to be treated is performed. A plurality of types of massage programs that are in accordance with the plurality of types of massage courses and a muscle hardness measurement program for executing the muscle hardness measurement process are stored in the memory 61.

The controller 60 includes a raising/lowering motor controller 62, an advancing/retreating motor controller 63, a kneading motor controller 64, and a tapping motor controller 65. The raising/lowering motor controller 62 controls the driving circuit 73 such that during driving of the raising/lowering motor 36, a rotation speed (rotation rate) of the raising/lowering motor 36 is a predetermined constant speed. The advancing/retreating motor controller 63 controls the driving circuit 74 such that during driving of the advancing/retreating motor 43, a rotation speed (rotation rate) of the advancing/retreating motor 43 is a predetermined constant speed. The kneading motor controller 64 controls the driving circuit 75 such that during driving of the kneading motor 48, a rotation speed (rotation rate) of the kneading motor 48 is a predetermined constant speed. The tapping motor controller 65 controls the driving circuit 76 such that during driving of the tapping motor 49, a rotation speed (rotation rate) of the tapping motor 49 is a predetermined constant speed.

The respective motors 36, 43, 48, and 49 are controlled (constant-speed controlled) such that the rotation speeds thereof are constant speeds and therefore motor currents increases when motor loads increase and the motor currents decrease when the motor loads decrease. That is, the currents of these motors change in accordance with magnitudes of the motor loads. The harder the muscle of the person to be treated, the greater the motor loads of the kneading motor 48 and the tapping motor 49. A magnitude (or average value) of the motor current of the kneading motor 48 or the tapping motor 49 is thus considered to be of a value that is in accordance with the hardness of the muscle of the person to be treated.

Also, the controller 60 includes an advancing/retreating motor current calculating portion 66, a kneading motor current calculating portion 67, and a tapping motor current calculating portion 68. The advancing/retreating motor current calculating portion 66 is a calculating portion arranged to calculate the motor current flowing through the advancing/retreating motor 43. The kneading motor current calculating portion 67 is a calculating portion arranged to calculate the motor current flowing through the kneading motor 48. The tapping motor current calculating portion 68 is a calculating portion arranged to calculate the motor current flowing through the tapping motor 49.

Further, the controller 60 includes a massage process portion 69 and a muscle hardness measurement process portion 70. The massage process portion 69 is a process portion, which, as is well known, performs the massage process in accordance with the massage mode (automatic mode or manual mode) and the massage course selected by the person to be treated. The muscle hardness measurement process portion 70 is a process portion that performs the muscle hardness measurement process in accordance with the muscle hardness measurement program inside the memory 61.

The raising/lowering motor controller 62, the advancing/retreating motor controller 63, the kneading motor controller 64, the tapping motor controller 65, the advancing/retreating motor current calculating portion 66, the kneading motor current calculating portion 67, the tapping motor current calculating portion 68, and the muscle hardness measurement process portion 70 shall now be described in detail.

First, the advancing/retreating motor controller 63 and the advancing/retreating motor current calculating portion 66 shall be described.

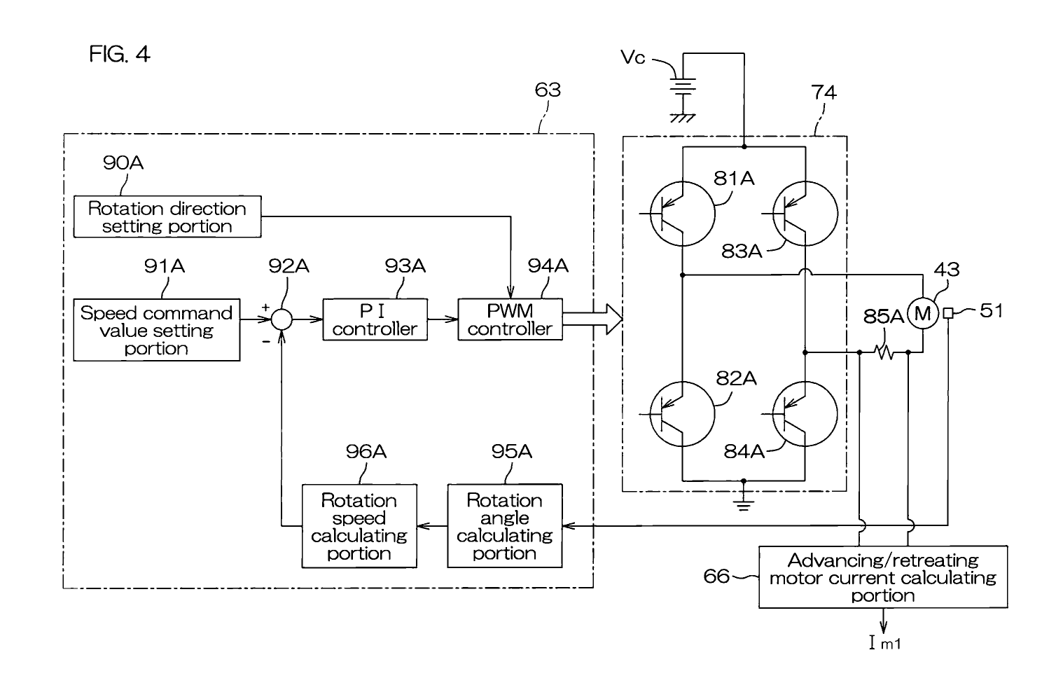

FIG. 4 is a block diagram of arrangements of the driving circuit 74 of the advancing/retreating motor 43 and the advancing/retreating motor controller 63 and showing the advancing/retreating motor current calculating portion 66.

In the present preferred embodiment, the advancing/retreating motor 43 is constituted of a DC motor with brush. The driving circuit 74 is constituted of an H bridge circuit that includes four, i.e., first to fourth switching elements 81A to 84A. Each of the switching elements 81A to 84A is constituted, for example, of a transistor. Specifically, the driving circuit 74 includes a series circuit, constituted of the high side first switching element 81A and the low side second switching element 82A, and a series circuit, constituted of the high side third switching element 83A and the low side fourth switching element 84A. Collectors of the high side switching elements 81A and 83A of the respective series circuits are connected to a positive electrode terminal of a power supply Vc. An emitter of the first switching element 81A is connected to a collector of the second switching element 82A. An emitter of the third switching element 83A is connected to a collector of the fourth switching element 84A. Emitters of the low side switching elements 82A and 84A of the respective series circuits are grounded.

A first terminal of the advancing/retreating motor 43 is connected to a connection point of the first switching element 81A and the second switching element 82A. A connection point of the third switching element 83A and the fourth switching element 84A is connected to a second terminal of the advancing/retreating motor 43 via a shunt resistor 85A. The shunt resistor 85A is a current detection resistor arranged to detect the motor current flowing through the advancing/retreating motor 43. The advancing/retreating motor current calculating portion 66 measures a voltage across both ends of the shunt resistor 85A to detect the motor current Im1 flowing through the advancing/retreating motor 43.

When the second switching element 82A and the third switching element 83A are turned off and the first switching element 81A and the fourth switching element 84A are turned on, the advancing/retreating motor 43 is rotated, for example, in a forward rotation direction. When the first switching element 81A and the fourth switching element 84A are turned off and the second switching element 82A and the third switching element 83A are turned on, the advancing/retreating motor 43 is rotated, for example, in a reverse rotation direction.

The advancing/retreating motor controller 63 includes a rotation direction setting portion 90A, a speed command value setting portion 91A, a speed deviation calculating portion 92A, a PI controller 93A, a PWM controller 94A, a rotation angle calculating portion 95A, and a rotation speed calculating portion 96A. The rotation angle calculating portion 95A calculates a rotor rotation angle of the advancing/retreating motor 43 based on an output signal of the rotation angle sensor 51. The rotation speed calculating portion 96A performs time differentiation of the rotor rotation angle of the advancing/retreating motor 43 calculated by the rotation angle calculating portion 95A to calculate the rotation speed of the advancing/retreating motor 43.

The rotation direction setting portion 90A sets a rotation direction command value that is in accordance with a direction (forward direction or backward direction) in which the massage element driving unit 40 is to be moved. The rotation direction command value set by the rotation direction setting portion 90A is provided to the PWM controller 94A. The speed command value setting portion 91A sets a rotation speed command value of the advancing/retreating motor 43. In the present preferred embodiment, the rotation speed command value is a predetermined constant value.

The speed deviation calculating portion 92A calculates a deviation (speed deviation) between the rotation speed command value set by the speed command value setting portion 91A and the rotation speed calculated by the rotation speed calculating portion 96A. The PI controller 93A performs PI calculation (proportional integration calculation) with the speed deviation calculated by the speed deviation calculating portion 92A to calculate a voltage command value.

Based on the rotation direction command value provided from the rotation direction setting portion 90A and the voltage command value provided from the PI controller 93A, the PWM controller 94A generates first and fourth PWM signals, provided respectively to the first and fourth switching elements 81A and 84A, and third and second PWM signals, provided respectively to the third and second switching elements 83A and 82A, and provides the signals to the driving circuit 74. If the rotation direction command value is a command value expressing the forward rotation direction, the first and fourth PWM signals are generated as PWM signals of duty ratios that are in accordance with the voltage command value, and the third and second PWM signals become inactive signals. In this case, the fourth PWM signal may be an H level signal instead of the PWM signal of the duty ratio that is in accordance with the voltage command value.

On the other hand, if the rotation direction command value is a command value expressing the reverse rotation direction, the third and second PWM signals are generated as PWM signals of duty ratios that are in accordance with the voltage command value, and the first and fourth PWM signals become inactive signals. In this case, the second PWM signal may be an H level signal instead of the PWM signal of the duty ratio that is in accordance with the voltage command value.

The driving circuit 74 controls the first to fourth switching elements 81A to 84A based on the first to fourth PWM signals provided from the PWM controller 94A. The advancing/retreating motor 43 is thereby driven to rotate such that the rotation direction is the rotation direction set by the rotation direction setting portion 90A and the rotation speed is equal to the rotation speed (predetermined constant speed) set by the rotation command value setting portion 91A.

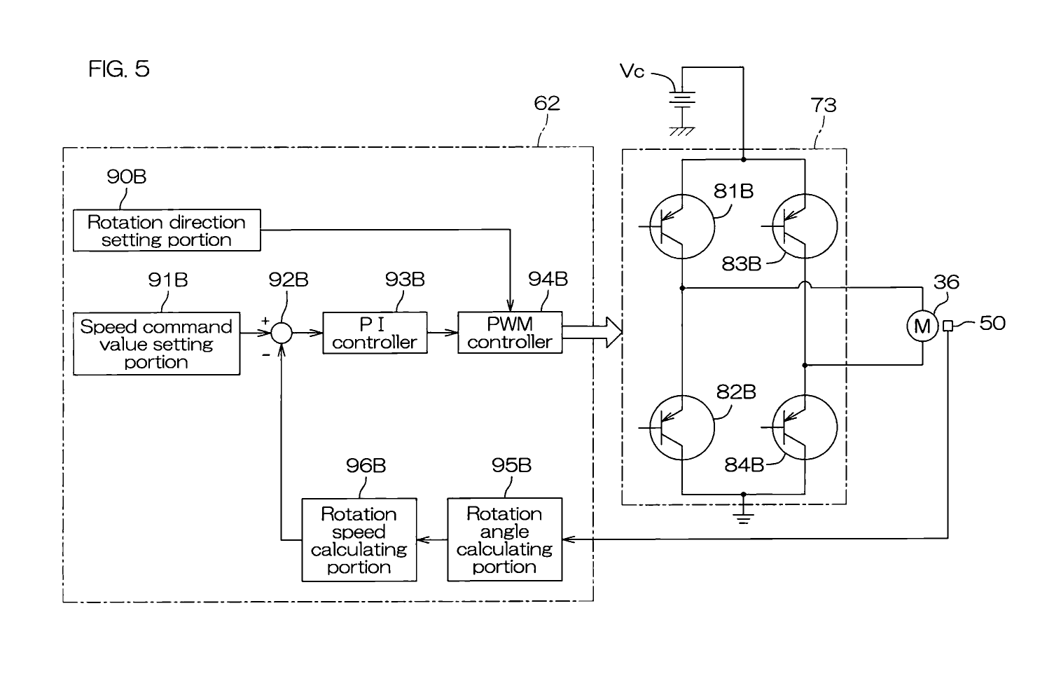

FIG. 5 is a block diagram of arrangements of the driving circuit 73 of the raising/lowering motor 36 and the raising/lowering motor controller 62. In FIG. 5, portions corresponding to the respective portions indicated by 81A to 84A and 90A to 96A in FIG. 4 are provided with symbols 81B to 84B and 90B to 96B.

In the present preferred embodiment, the raising/lowering motor 36 is constituted of a DC motor with brush. The driving circuit 73 has the same arrangement as the driving circuit 74 of the advancing/retreating motor 43 described above and is constituted of an H bridge circuit that includes four, i.e., first to fourth switching elements 81B to 84B. The four, i.e., first to fourth switching elements 81B to 84B correspond to the four, i.e., first to fourth switching elements 81A to 84A of FIG. 4 described above. However, in the present preferred embodiment, the driving circuit 73 is not provided with a shunt resistor. Each of the switching elements 81B to 84B is constituted, for example, of a transistor. A first terminal of the raising/lowering motor 36 is connected to a connection point of the first switching element 81B and the second switching element 82B. A connection point of the third switching element 83B and the fourth switching element 84B is connected to a second terminal of the raising/lowering motor 36.

The raising/lowering motor controller 62 has the same arrangement as the above-described advancing/retreating motor controller 63 of FIG. 4 and includes a rotation direction setting portion 90B, a speed command value setting portion 91B, a speed deviation calculating portion 92B, a PI controller 93B, a PWM controller 94B, a rotation angle calculating portion 95B, and a rotation speed calculating portion 96B. The rotation direction setting portion 90B sets a rotation direction command value that is in accordance with a direction (raising direction or lowering direction) in which the massage unit 17 is to be moved. Operations of the other respective portions 91B to 96B of FIG. 5 are the same as those of the respective corresponding portions 91A to 96A of FIG. 4 and therefore description thereof shall be omitted.

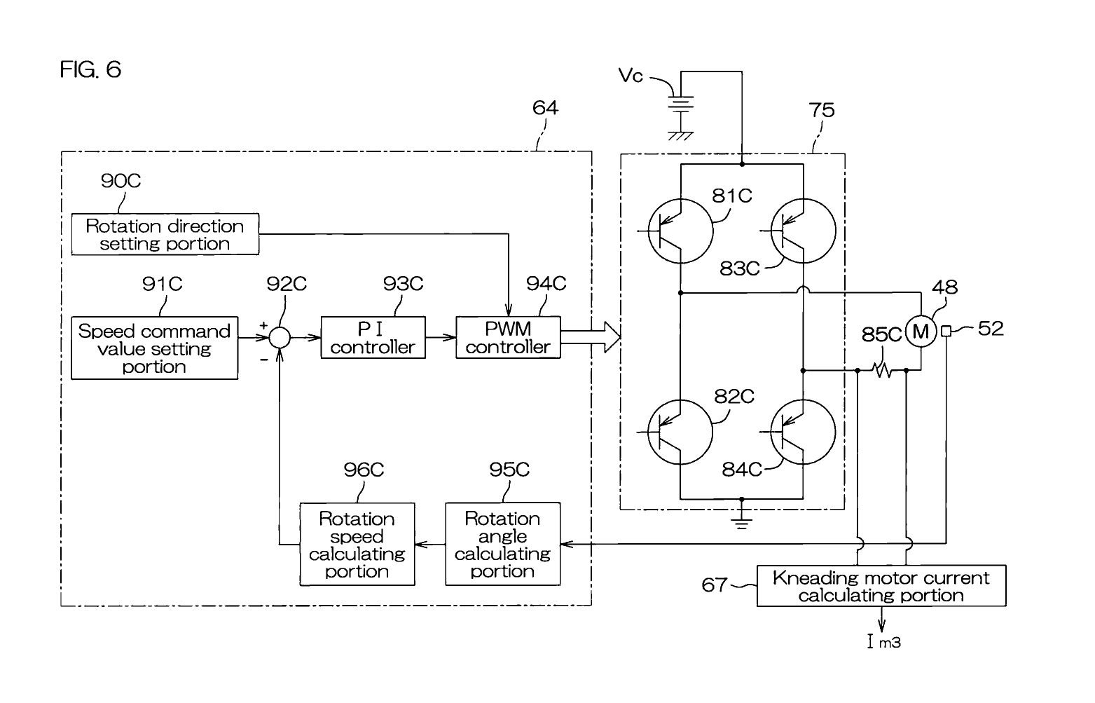

FIG. 6 is a block diagram of arrangements of the driving circuit 75 of the kneading motor 48 and the kneading motor controller 64 and showing the kneading motor current calculating portion 67. In FIG. 6, portions corresponding to the respective portions indicated by 81A to 85A and 90A to 96A in FIG. 4 are provided with symbols 81C to 85C and 90C to 96C.

In the present preferred embodiment, the kneading motor 48 is constituted of a DC motor with brush. The driving circuit 75 has the same arrangement as the driving circuit 74 of the advancing/retreating motor 43 described above and is constituted of an H bridge circuit that includes four, i.e., first to fourth switching elements 81C to 84C. The four, i.e., first to fourth switching elements 81C to 84C correspond to the four, i.e., first to fourth switching elements 81A to 84A of FIG. 4 described above. Each of the switching elements 81C to 84C is constituted, for example, of a transistor. A first terminal of the kneading motor 48 is connected to a connection point of the first switching element 81C and the second switching element 82C. A connection point of the third switching element 83C and the fourth switching element 84C is connected to a second terminal of the kneading motor 48 via a shunt resistor 85C. The shunt resistor 85C is a current detection resistor arranged to detect the motor current flowing through the kneading motor 48. The kneading motor current calculating portion 67 measures a voltage across both ends of the shunt resistor 85C to detect the motor current Im3 flowing through the kneading motor 48.

The kneading motor controller 64 has the same arrangement as the above-described advancing/retreating motor controller 63 of FIG. 4 and includes a rotation direction setting portion 90C, a speed command value setting portion 91C, a speed deviation calculating portion 92C, a PI controller 93C, a PWM controller 94C, a rotation angle calculating portion 95C, and a rotation speed calculating portion 96C. The rotation direction setting portion 90C sets a rotation direction command value that is in accordance with the type of kneading operation (kneading up or kneading down) that is to be executed. Operations of the other respective portions 91C to 96C of FIG. 6 are the same as those of the respective corresponding portions 91A to 96A of FIG. 4 and therefore description thereof shall be omitted.

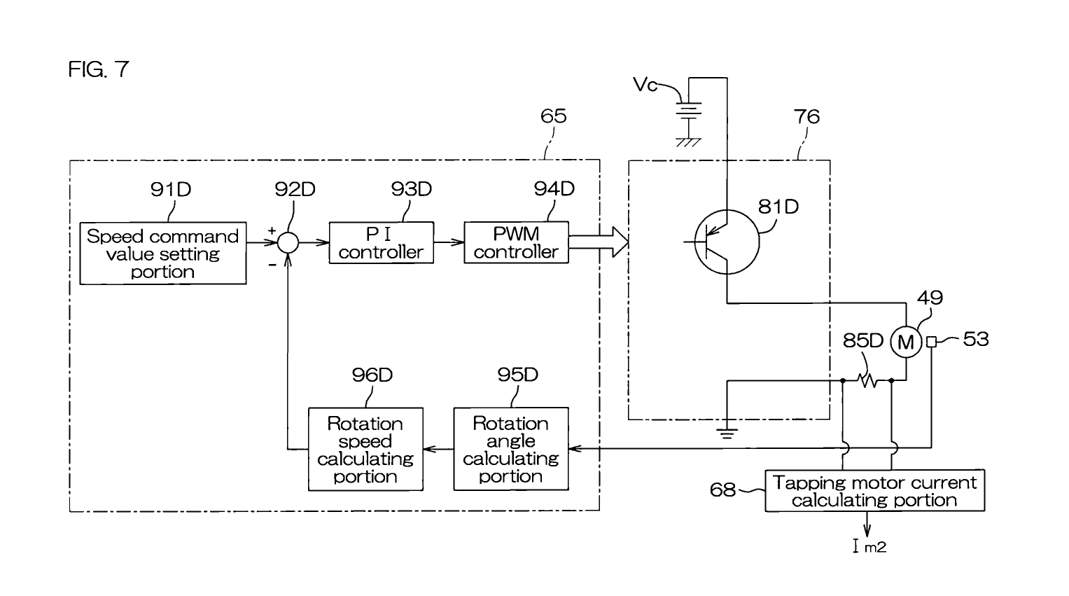

FIG. 7 is a block diagram showing arrangements of the driving circuit 76 of the tapping motor 49 and the tapping motor controller 65 and showing the tapping motor current calculating portion 68. In FIG. 7, portions corresponding to the respective portions indicated by 91A to 96A in FIG. 4 are provided with symbols 91D to 96D.

In the present preferred embodiment, the tapping motor 49 is constituted of a DC motor with brush. The driving circuit 76 of the tapping motor 49 includes a switching element 81D. The switching element 81D is constituted, for example, of a transistor. A collector of the switching element 81D is connected to the positive electrode terminal of a power supply Vc. An emitter of the switching element 81D is connected to a first terminal of the tapping motor 49. A second terminal of the tapping motor 49 is grounded via a shunt resistor 85D. The shunt resistor 85D is a current detection resistor arranged to detect the motor current flowing through the tapping motor 49. The tapping motor current calculating portion 68 measures a voltage across both ends of the shunt resistor 85D to detect the motor current Im2 flowing through the tapping motor 49.

The tapping motor controller 65 includes a speed command value setting portion 91D, a speed deviation calculating portion 92D, a PI controller 93D, a PWM controller 94D, a rotation angle calculating portion 95D, and a rotation speed calculating portion 96D. Operations of the speed command value setting portion 91D, the speed deviation calculating portion 92D, the PI controller 93D, the rotation angle calculating portion 95D, and the rotation speed calculating portion 96D are the same as those of the speed command value setting portion 91A, the speed deviation calculating portion 92A, the PI controller 93A, the rotation angle calculating portion 95A, and the rotation speed calculating portion 96A of the advancing/retreating motor controller 63. Based on a voltage command value provided from the PI controller 93D, the PWM controller 94D generates a PWM signal of a duty ratio that is in accordance with the voltage command value and provides the signal to the driving circuit 76. The driving circuit 76 controls the switching element 81D based on the PWM signal provided from the PWM controller 94D. The tapping motor 49 is thereby driven to rotate such that the rotation speed is equal to a rotation speed (predetermined constant speed) set by the speed command value setting portion 91D.

Next, the muscle hardness measurement process portion 70 shall be described. When muscle hardness is desired to be measured, the person to be treated first operates the operating device 23 to move the massage elements 41 to a portion at which the muscle hardness is desired to be measured (portion to be measured, for example, a shoulder portion). Thereafter, the person to be treated operates the operating device 23 to select the muscle hardness measurement mode. When the muscle hardness measurement mode is selected, the muscle hardness measurement process portion 70 starts the muscle hardness measurement process.

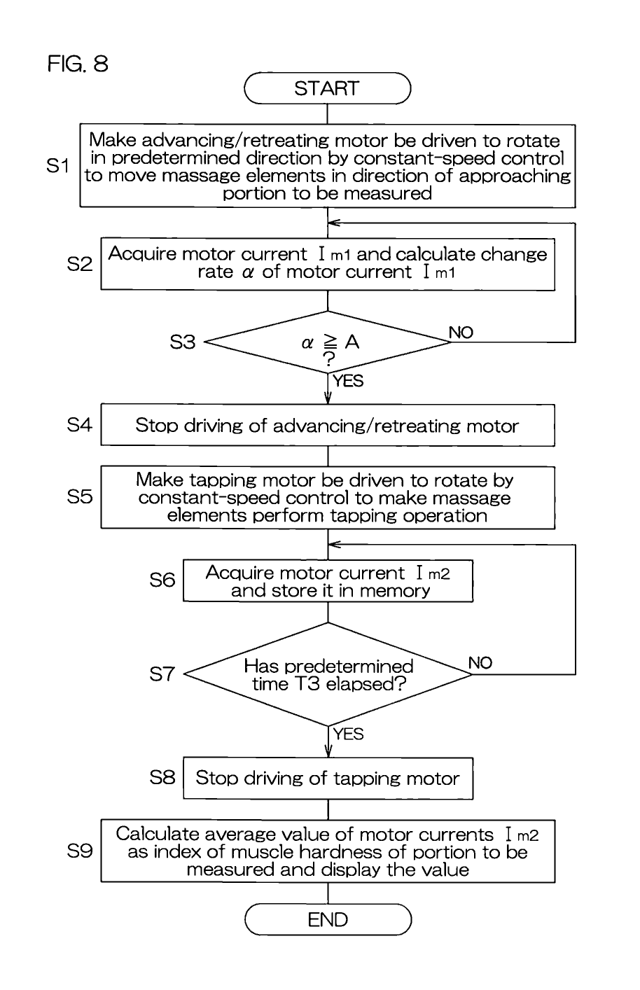

FIG. 8 is a flowchart of procedures of the muscle hardness measurement process executed by the muscle hardness measurement process portion 70.

When the muscle hardness measurement mode is selected, the muscle hardness measurement process portion 70 first makes the advancing/retreating motor 43 be driven to rotate in a predetermined direction by constant-speed control via the advancing/retreating motor controller 63 to move the massage elements 41 (massage element driving unit 40) in a direction of approaching the portion to be measured of the person to be treated (step S1). Thereby, the massage elements 41 advance and are pressed against the portion to be measured.

When the driving of the advancing/retreating motor 43 is started, the muscle hardness measurement process portion 70 repeatedly performs processes of subsequent steps S2 and S3 every first predetermined time T1 until a judgment result of step S3 becomes a positive judgment. In step S2, the muscle hardness measurement process portion 70 acquires the motor current Im1 calculated by the advancing/retreating motor current calculating portion 66 and performs time differentiation of the acquired motor current Im1 to calculate a change rate α of the motor current Im1.

If Im1(n) is the motor current Im1 acquired currently and Im1(n−1) is the motor current Im1 acquired previously (at the predetermined time before), the change rate α of the motor current Im1 is expressed by the following formula (1).

α=Im 1(n)−Im 1(n−1) (1)

In step S3, the muscle hardness measurement process portion 70 determines whether or not the change rate α of the motor current Im1 calculated in step S2 is not less than a predetermined threshold A (A>0). A reason for making such judgment shall now be described. A hardness of a body is a hardness due to a multilayer structure of skin, fat, and muscle. There are individual differences in thicknesses and hardnesses of skin and fat and therefore to measure a hardness of just a muscle, the hardness of the muscle must be measured after the massage elements 41 are pressed in to a position at which the skin and fat cannot be compressed any further.

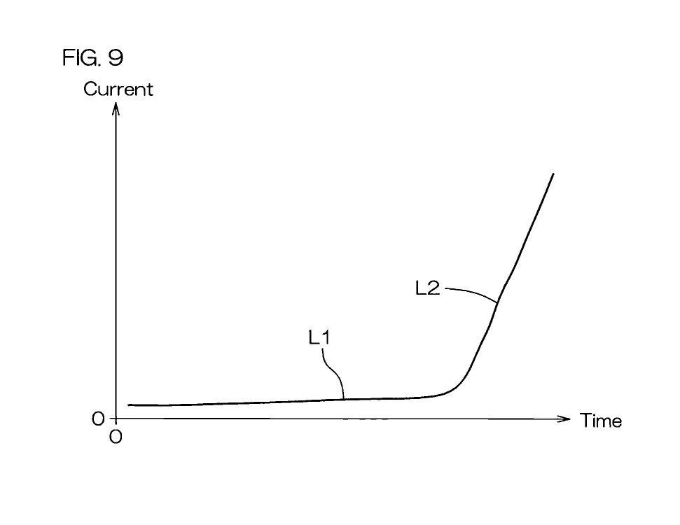

FIG. 9 is a graph of change with time of the motor current Im1 flowing through the advancing/retreating motor 43 when the massage elements 41 are pressed into the body (portion to be measured) at the constant speed by the advancing/retreating motor 43.

As the massage elements 41 are pressed into the body at the constant speed from a state of contacting a surface of the body, first, the skin and fat covering a surface of the muscle is compressed by the massage elements 41. When the massage elements 41 are pressed in to the position at which the skin and fat cannot be compressed any further, the muscle becomes compressed by the massage elements 41. The muscle is hard in comparison to the skin and fat and therefore, the change rate of the motor current Im1 when the massage elements 41 are pressed into the body at the constant speed is such that the change rate α for the muscle is large in comparison to the change rate for the skin and fat.

It is therefore considered that in the graph of FIG. 9, an initial gradual rectilinear portion L1 indicates the change of the motor current Im1 when the skin and fat are being compressed by the massage elements 41 and a steep rectilinear portion L2 following thereafter via a curve portion indicates the change of the motor current Im1 when the muscle is being compressed by the massage elements 41. From this, it may be considered that a slope of the rectilinear portion L1 corresponds to the change rate α (referred to hereinafter as α1) of the motor current Im1 for the skin and fat and a slope of the rectilinear portion L2 of greater slope than the rectilinear portion L1 corresponds to the change rate α (referred to hereinafter as α2) of the motor current Im1 for the muscle.

The judgment in step S3 is made to judge whether or not the massage elements 41 have been pressed in to the position at which the skin and fat of the portion to be measured cannot be compressed any further by the massage elements 41. The threshold A is set, for example, to a value in between the slope α1 of the rectilinear portion L1 and the slope α2 of the rectilinear portion L2.

If in step S3, it is determined that the change rate α of the motor current Im1 is less than the threshold A (step S3: NO), the muscle hardness measurement process portion 70 returns to step S2 and performs the processes of steps S2 and S3 again. In this case, a starting timing of the current step S2 process is controlled such that the current step S2 process is executed when the first predetermined time T1 has elapsed from when the previous step S2 process was performed.

If in step S3, it is determined that the change rate α of the motor current Im1 is not less than the threshold A (step S3: YES), the muscle hardness measurement process portion 70 stops the driving of the advancing/retreating motor 43 (step S4).

Next, the muscle hardness measurement process portion 70 makes the tapping motor 49 be driven to rotate by constant-speed control via the tapping motor controller 65 to make the massage elements 41 perform the tapping operation (step S5). Thereby, the tapping operation is performed on the portion to be measured.

When the driving of the tapping motor 49 is started, the muscle hardness measurement process portion 70 repeatedly performs processes of subsequent steps S6 and S7 every second predetermined time T2 until a judgment result of step S7 becomes a positive judgment. In step S6, the muscle hardness measurement process portion 70 acquires the motor current Im2 calculated by the tapping motor current calculating portion 68 and stores it in the memory 61.

In step S7, the muscle hardness measurement process portion 70 determines whether or not a third predetermined time T3 has elapsed since the tapping motor 49 is driven to rotate in step S5. If the third predetermined time T3 has not elapsed (step S7: NO), the muscle hardness measurement process portion 70 returns to step S6 and executes the processes of steps S6 and S7 again. In this case, a starting timing of the current step S6 process is controlled such that the current step S6 process is executed when the second predetermined time T2 has elapsed from when the previous step S6 process was performed.

If in step S7, it is determined that the third predetermined time T3 has elapsed (step S7: YES), the muscle hardness measurement process portion 70 stops the driving of the tapping motor 49 (step S8).

Next, the muscle hardness measurement process portion 70 calculates an average value of a plurality of motor currents Im2 stored in the memory 61 from when the tapping motor 49 is driven to rotate in step S5 to when it is stopped in step S8 as an index of the muscle hardness of the portion to be measured of the person to be treated and displays the value on the display device 24 (step S9). The muscle hardness measurement process portion 70 then ends the current muscle hardness measurement process.

In step S9, an average value of a plurality of motor currents Im2, which, among the plurality of motor currents Im2 stored in the memory 61 from when the tapping motor 49 is driven to rotate in step S5 to when it is stopped in step S8, are stored in the memory 61 in a fourth predetermined time T4 shorter than the third predetermined time T3, may be calculated instead as the index of the muscle hardness of the portion to be measured of the person to be treated.

With the present preferred embodiment, in the muscle hardness measurement mode, after the massage elements 41 are pressed in by the advancing/retreating motor 43 to the position at which the skin and fat of the portion to be measured cannot be compressed any further, the tapping motor 49 can be driven to rotate by constant-speed control to perform the tapping operation using the massage elements 41. The muscle hardness of the portion to be measured can then be measured based on the motor currents of the tapping motor 49 detected during the tapping operation. It is thereby made possible to measure the hardness of just the muscle.

Also, the tapping motor 49 is the driving source of the tapping mechanism, arranged to make the massage elements 41 perform the tapping operation, and the advancing/retreating motor 43 is a driving source of the massage element driving unit advancing/retreating mechanism (moving mechanism), arranged to move the massage element driving unit 40, including the tapping mechanism, in the approaching/separating direction with respect to the person to be treated. That is, the tapping motor 49 and the advancing/retreating motor 43 are motors that are inherently included in the chair type massage machine 1 for performing the tapping operation on the person to be treated. Therefore, with the present preferred embodiment, the hardness of just the muscle can be measured without using special measurement equipment.

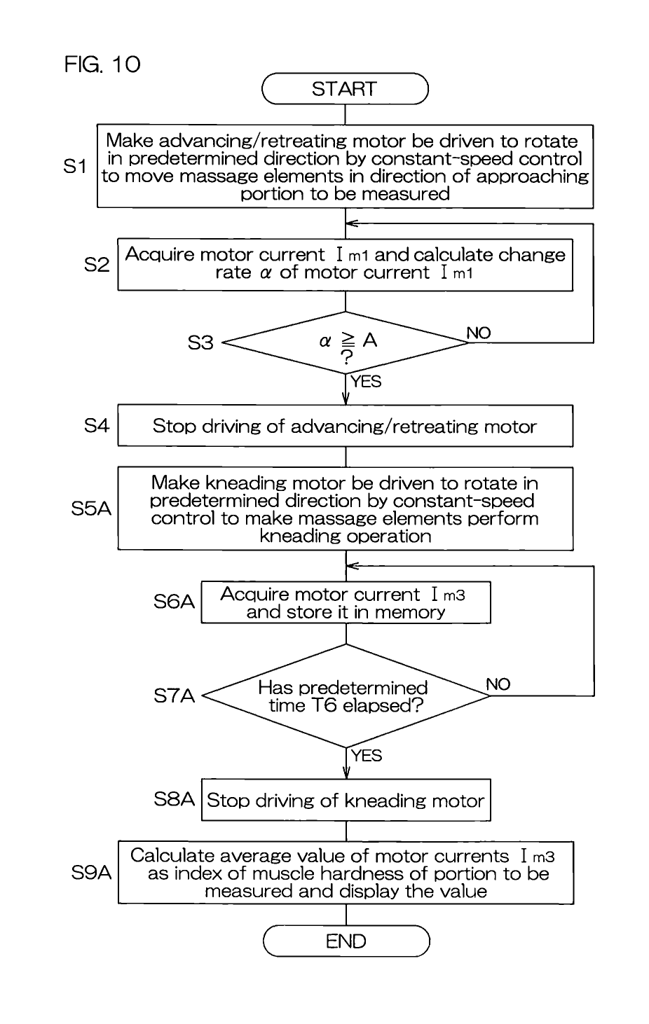

FIG. 10 is a flowchart of procedures of another example of the muscle hardness measurement process executed by the muscle hardness measurement process portion 70. In FIG. 10, steps corresponding to steps of respective portions of FIG. 8 described above are provided and indicated with the same step numbers.

The processes of steps S1 to S4 of FIG. 10 are the same as the processes of steps S1 to S4 of FIG. 8 and therefore description thereof shall be omitted.

When the driving of the advancing/retreating motor 43 is stopped in step S4, the muscle hardness measurement process portion 70 makes the kneading motor 48 be driven to rotate in a predetermined direction (for example, the kneading up operation direction) by constant-speed control via the kneading motor controller 64 to make the massage elements 41 perform the kneading operation (for example, the kneading up operation) (step S5A). Thereby, the kneading operation (for example, the kneading up operation) is performed on the portion to be measured.

When the driving of the kneading motor 48 is started, the muscle hardness measurement process portion 70 repeatedly performs processes of subsequent steps S6A and S7A every fifth predetermined time T5 until a judgment result of step S7A becomes a positive judgment. In step S6A, the muscle hardness measurement process portion 70 acquires the motor current Im3 calculated by the kneading motor current calculating portion 67 and stores it in the memory 61.

In step S7A, the muscle hardness measurement process portion 70 determines whether or not a sixth predetermined time T6 has elapsed since the kneading motor 48 is driven to rotate in step S5A. If the sixth predetermined time T6 has not elapsed (step S7A: NO), the muscle hardness measurement process portion 70 returns to step S6A and executes the processes of steps S6A and S7A again. In this case, a starting timing of the current step S6A process is controlled such that the current step S6A process is executed when the fifth predetermined time T5 has elapsed from when the previous step S6A process was performed.

If in step S7A, it is determined that the sixth predetermined time T6 has elapsed (step S7A: YES), the muscle hardness measurement process portion 70 stops the rotation driving of the kneading motor 48 (step S8A).

Next, the muscle hardness measurement process portion 70 calculates an average value of a plurality of motor currents Im3 acquired from the kneading motor current calculating portion 67 and stored in the memory 61 from when the kneading motor 48 is driven to rotate in step S5A to when it is stopped in step S8A as an index of the muscle hardness of the portion to be measured of the person to be treated and displays the value on the display device 24 (step S9A). The muscle hardness measurement process portion 70 then ends the current muscle hardness measurement process.

In step S9A, an average value of a plurality of motor currents Im3, which, among the plurality of motor currents Im3 stored in the memory 61 from when the kneading motor 48 is driven to rotate in step S5A to when it is stopped in step S8A, are stored in the memory 61 in a seventh predetermined time T7 shorter than the sixth predetermined time T6, may be calculated instead as the index of the muscle hardness of the portion to be measured of the person to be treated.

With the modification example of FIG. 10, in the muscle hardness measurement mode, after the massage elements 41 are pressed in by the advancing/retreating motor 43 to the position at which the skin and fat of the portion to be measured cannot be compressed any further, the kneading motor 48 can be driven to rotate by constant-speed control to perform the kneading operation using the massage elements 41. The muscle hardness of the portion to be measured can then be measured based on the motor currents of the kneading motor 48 detected during the kneading operation. It is thereby made possible to measure the hardness of just the muscle.

Also, the kneading motor 48 is the driving source of the kneading mechanism, arranged to make the massage elements 41 perform the kneading operation, and the advancing/retreating motor 43 is the driving source of the massage element driving unit advancing/retreating mechanism (moving mechanism), arranged to move the massage element driving unit 40, including the kneading mechanism, in the approaching/separating direction with respect to the person to be treated. That is, the kneading motor 48 and the advancing/retreating motor 43 are motors that are inherently included in the chair type massage machine 1 for performing the kneading operation on the person to be treated. Therefore, with the present preferred embodiment, the hardness of just the muscle can be measured without using special measurement equipment.

Although a preferred embodiment of the present invention has been described above, the present invention may also be implemented in yet other modes. For example, although in the preferred embodiment described above, the raising/lowering motor 36, the advancing/retreating motor 43, the kneading motor 48, and the tapping motor 49 are DC motors with brushes, these motors 36, 43, 48, and 49 may instead be electric motors of another type, such as three-phase brushless motors, etc. In a case where these motors are three-phase brushless motors, three-phase inverter circuits are used as driving circuits thereof.

Also, the operating device 23 may include, for example, a display device or a touch panel type display device. In this case, the display device 24 may be omitted.

Also, although with the preferred embodiment described above, a case where the present invention is applied to a chair type massage machine was described, the present invention may be applied to a massage machine other than a chair type (for example, a seat type massage machine) as long as it is a massage machine that includes a tapping mechanism or a kneading mechanism having an electric motor as a driving source. Also, the present invention may be applied to a massage machine that does not include an air bag.

The present application corresponds to Japanese Patent Application No. 2017-201210 filed on Oct. 17, 2017 in the Japan Patent Office, and the entire disclosure of this application is incorporated herein by reference.

While preferred embodiments of the present invention have been described above, it is to be understood that variations and modifications will be apparent to those skilled in the art without departing the scope and sprit of the present invention. The scope of the present invention, therefore, is to be determined solely by the following claims.

What is claimed is:

1. A massage machine having a muscle hardness measurement mode of measuring a muscle hardness of a person to be treated, the massage machine comprising:
a massage element driving unit, having a first electric motor as a driving source and including a massage mechanism arranged to make a massage element perform a massage operation;
a moving mechanism, having a second electric motor as a driving source and arranged to move the massage element driving unit in an approaching/separating direction with respect to a body of the person to be treated;
a second electric motor driving portion, which, in the muscle hardness measurement mode, drives the second electric motor to rotate in a predetermined direction by constant-speed control to press the massage element into the body of the person to be treated;
a second electric motor drive stopping portion, monitoring a change rate of a motor current flowing through the second electric motor during driving of the second electric motor by the second electric motor driving portion and stopping the driving of the second electric motor when the change rate of the motor current becomes not less than a predetermined threshold;
a first electric motor driving portion, which, after stoppage of the driving of the second electric motor by the second electric motor drive stopping portion, drives the first electric motor to rotate by constant-speed control to make the massage element perform the massage operation;
a current detection portion, detecting a motor current flowing through the first electric motor during driving of the first electric motor by the first electric motor driving portion; and
a muscle hardness measurement portion, measuring the muscle hardness of the person to be treated based on the motor current detected by the current detection portion.

2. The massage machine according to claim 1, wherein the muscle hardness measurement portion is arranged to calculate, as an index value of the hardness of the muscle of the person to be treated, an average value of the motor current detected by the current detection portion in a predetermined time during the driving of the first electric motor by the first electric motor driving portion.

3. The massage machine according to claim 1, wherein the massage mechanism is a tapping mechanism arranged to make the massage element perform a tapping operation.

4. The massage machine according to claim 1, wherein the massage mechanism is a kneading mechanism arranged to make the massage element perform a kneading operation.

5. The massage machine according to claim 2, wherein the massage mechanism is a tapping mechanism arranged to make the massage element perform a tapping operation.

6. The massage machine according to claim 2, wherein the massage mechanism is a kneading mechanism arranged to make the massage element perform a kneading operation.
